SEQUENCE LISTING (6 sequences)

SEQ ID NO: 1 - DNA, 38 bp, Artificial Sequence
  ApoA-I forward primer
caccaggagatatacatatgaaagctgcggtgctgacc

SEQ ID NO: 2 - DNA, 45 bp, Artificial Sequence
  ApoA-I reverse primer
ctagtggtggtggtggtggtgctgggtgttgagcttcttagtgta

SEQ ID NO: 3 - DNA, 38 bp, Artificial Sequence
  TAAR13c forward primer-1
caccaggagatatacatatgatgcccttttgccacaat

SEQ ID NO: 4 - DNA, 28 bp, Artificial Sequence
  TAAR13c reverse primer-1
tgaactcaattccaaaaataatttacac

SEQ ID NO: 5 - DNA, 30 bp, Artificial Sequence
  TAAR13c forward primer-2
atgaattcatggatttatcatcacaagaat

SEQ ID NO: 6 - DNA, 30 bp, Artificial Sequence
  TAAR13c reverse primer-2
atctcgagtcaaaccgtaaataaattgata

(19) United States
(12) Patent Application Publication
(10) Pub. No.: US 2021/0214401 A1
(43) Pub. Date: Jul. 15, 2021

(54) MANUFACTURING METHOD OF NANODISC COMPRISING AN OLFACTORY RECEPTOR PROTEIN AND NANODISC COMPRISING AN OLFACTORY RECEPTOR PROTEIN MANUFACTURED BY THE SAME

(71) Applicant: SEOUL NATIONAL UNIVERSITY R&DB FOUNDATION, Seoul (KR)

(72) Inventors: Tai Hyun Park, Seoul (KR); Heehong Yang, Yongin-si Gyeonggi-do (KR)

(73) Assignee: SEOUL NATIONAL UNIVERSITY R&DB FOUNDATION, Seoul (KR)

(21) Appl. No.: 16/174,759

(22) Filed: Oct. 30, 2018

(30) Foreign Application Priority Data
Nov. 3, 2017 (KR) 10-2017-0146128

(51) Int. Cl. C07K 14/245; C07K 14/775; C07K 17/02

(52) U.S. Cl. CPC C07K 14/245; C07K 17/02; C07K 14/775

(57) ABSTRACT

The present invention relates to a manufacturing method of a nanodisc comprising an olfactory receptor protein, and a nanodisc comprising an olfactory receptor protein manufactured by the same, and more specifically, a manufacturing method of a nanodisc comprising an olfactory receptor protein using E. coli, and a nanodisc comprising an olfactory receptor protein manufactured by the same.

According to the present invention, nanodiscs (T13NDs) are manufactured by producing receptors used in T13NDs from E. coli, thereby being able to mimic the original receptor structure and can be stable in water and atmospheric environments, and by the same, not only selectivity, accuracy, and reproducibility can be improved, but also it was possible to selectively detect cadaverine, which is known to occur from rotten foods, through the improved performance ability.

### BACKGROUND OF THE INVENTION

### Field of the Invention

The present invention relates to a manufacturing method of a nanodisc comprising an olfactory receptor protein, and a nanodisc comprising an olfactory receptor protein manufactured by the same, and more specifically, a manufacturing method of a nanodisc comprising an olfactory receptor protein produced in E. coli, and a nanodisc comprising an olfactory receptor protein manufactured by the same.

### Related Art

G protein-coupled receptors (GPCRs) play important roles in cell responses of the human body. Therefore, GPCRs are deeply involved in many people's disease, and thus approximately 40% of all modem medicines target the GPCRs. Trace amine-associated receptors (TAARs), classified as GPCRs, are common amine receptors that bind to endogenous compounds that are structurally related to conventional biogenic amines. Trace amine-associated receptor 13c (TAAR13c) is known to play a role in an olfactory receptor in zebrafish (Danio rerio), and it is also known to have a specific effect on cadaverine, which is an odor component associated with decomposition. Cadaverine, produced by the bacterial decarboxylation of lysine, is one of the various bioamines that emit extremely unpleasant odors to humans. In addition, due to the variety of foods that contain lysine, cadaverine is one of the most important markers for detecting rotten foods. Therefore, it is expected that TAAR13c can use a variety of fields such as industrial applications and scientific exploitation to detect cadaverine.

Among the many reconstitution techniques for receptors, nanodiscs (NDs) are considered to be the most suitable tool for GPCRs reconstruction. Each nanodisc consists of a receptor, a lipid bilayer, and a membrane scaffold protein (MSP), and the nanodisc comprising the GPCRs as a receptor is tightly surrounded at the edge of the lipid bilayer. Therefore, nanodiscs can be stable in water and atmospheric environments and can mimic the original structure of the receptor in cells. Conventionally, nanodisc-based biosensors using receptor expressed in SF9 cells are also well known.

For the production of recombinant proteins, E. coli, as a prokaryote, has been widely used as a host cell because of its advantages such as productivity and simplicity. However, when GPCRs are produced in E. coli, it is difficult to express GPCRs in E. coli, a prokaryotic cell, and there still remains a challenge to solve due to strong hydrophobicity, differences in charge distribution, and different membrane insertion mechanisms. GPCRs comprising olfactory receptors are a membrane protein of eukaryotic cells, and for their expression, they are generally expressed on a cell membrane using animal cells and insect cells. However, in this case, there is a disadvantage in that productivity and cost effectiveness (cell maintenance and expression cost) are lowered. When GPCRs are produced using E. coli, productivity and cost effectiveness are superior to using animal cells and insect cells. However, since E. coli is a prokaryotic cell, it is difficult to express the membrane proteins of GPCRs, and when GPCRs are unreasonably forced to be overexpressed there is a problem in that the E. coli will die.

In this regard, the present inventors have made efforts to overcome the problems of the conventional technologies. As a result, they have developed a manufacturing method of a novel nanodisc comprising an olfactory receptor protein, which is stable in water and atmospheric environments mimicking the original receptor structure, and manufactured nanodiscs produced by using an olfactory receptor protein from E. coli, thereby implementing the unique structure of the membrane protein by optimizing the secondary structure of the receptor, by securing the productivity and stability of the receptor protein and implementing the environment very similar to the biological environment. Additionally, they have confirmed that the method not only can improve selectivity, accuracy, and reproducibility, but also can selectively sense cadaverine from rotten foods by the improved performance ability, and distinguish the degree of decomposition of foods, thereby completing the present invention.

### SUMMARY OF THE INVENTION

An object of the present invention is to provide a manufacturing method of a nanodisc comprising an olfactory receptor protein, which is stable in water and atmospheric environments mimicking the original receptor structure by comprising an olfactory receptor protein produced in E. coli.

Another object of the present invention is to provide a nanodisc comprising an olfactory receptor protein manufactured using the manufacturing method of a nanodisc described above.

According to an aspect of the present invention, the present invention provides a manufacturing method of a nanodisc comprising an olfactory receptor protein, which consists:

i) producing and purifying an olfactory receptor protein in E. coli;

ii) producing and purifying a membrane scaffold protein in E. coli;

iii) mixing the olfactory receptor protein, which was produced and purified in E. coli, with lipids, followed by settling;

iv) mixing the settled mixture with the membrane scaffold protein, which was produced and purified in E. coli, and stirring, thereby assembling a nanodisc; and

v) removing surfactants and unbound proteins from the mixture of iv).

In the manufacturing method of a nanodisc comprising an olfactory receptor protein, the producing of an olfactory receptor protein in E. coli in step i) comprises:

i-1) culturing the E. coli transformed with an olfactory receptor protein;

i-2) overexpressing an olfactory receptor protein;

i-3) lysing the E. coli and releasing the olfactory receptor protein to the outside of the cell; and

i-4) solubilizing the olfactory receptor protein, followed by separation and purification.

In the present invention, the E. coli transformed with an olfactory receptor protein is cultured to a certain concentration or above, and the olfactory receptor protein is overexpressed in the cultured E. coli. The cultured E. coli is then lysed and the overexpressed olfactory receptor protein is released to the outside of the cell. The released olfactory receptor protein is dissolved using a surfactant, etc., separated and purified, and mixed well with a membrane scaffold protein and a lipid to be reconstituted into a nanodisc, thereby mimicking the original receptor structure such that it is stable in water and atmospheric environments.

In the present invention, the olfactory receptor protein is characterized in that it is expressed in the form of an inclusion body within E. coli, and the protein in the form of an inclusion body expressed by the lysing E. coli is released to the outside of the cell, and the protein in the form of an inclusion body is dissociated by mixing with a surfactant, etc. then, the resultant is purified and again reconstituted in the form of an olfactory receptor protein. That is, the present inventors mass-produced an olfactory receptor protein in the form of an inclusion body within E. coli, and released the olfactory receptor protein overexpressed within E. coli by the lysis of E. coli to the outside of the cell, dissociated the inclusion body, and produced an olfactory receptor protein through the reconstitution process using E. coli.

In the manufacturing method of a nanodisc comprising an olfactory receptor protein by the present invention, the olfactory receptor protein is characterized in that it includes, without limitation, receptors associated with hormones, olfactory receptors, taste buds, and neurotransmitters, and more preferably, it is the trace amine-associated receptor 13c (TAAR13c) protein that specifically reacts to cadaverine.

In the manufacturing method of a nanodisc comprising an olfactory receptor protein by the present invention, the cadaverine is a component of ptomaine caused by the decomposition of meat or other proteins, and it emits a rotten odor but is not toxic. Additionally, it is a degradation product of lysine and is produced along with putrescine due to the decomposition of ptomaine protein.

In the manufacturing method of a nanodisc comprising an olfactory receptor protein by the present invention, as the membrane scaffold protein, any protein that added to encompass the lipid-receptor complex, can be used, and preferably the apolipoprotein A-I (ApoA-I) protein may be used.

In the manufacturing method of a nanodisc comprising an olfactory receptor protein by the present invention, in step iii) where the olfactory receptor protein produced in E. coli is mixed with lipids, followed by settling, the stirring is performed at 0° C. to 10° C. for 10 minutes to 1 hour.

In the manufacturing method of a nanodisc comprising an olfactory receptor protein by the present invention, in step iii) where the olfactory receptor protein produced in E. coli is mixed with lipids, followed by settling, I-palmitoyl-2-oleoyl-sn-glycero-3-phosphocholine (POPC) and 1-palmitoyl-2-oleoyl-sn-glycero-3-phosphoglycerol (POPG) are mixed in a 1:1 molecular rate, so as to provide an environment similar to the membrane environment.

In the manufacturing method of a nanodisc comprising an olfactory receptor protein by the present invention, in step iv) where the purified membrane scaffold protein is added thereto and mixed, and stirred thereby assembling a nanodisc, the stirring is performed for 1 to 2 hours.

The present invention also provides a nanodisc comprising an olfactory receptor protein, which is manufactured by the method of the present invention, and has an average diameter of 15 nm or greater, and more preferably 15 nm to 25 nm.

The structure of the nanodisc manufactured in the present invention is shown in FIG. 1. As shown in FIG. 1, the nanodisc manufactured in the present invention forms a nanodisc structure by surrounding the olfactory receptor protein with a membrane scaffold protein in E. coli.

According to an embodiment of the present invention, the size of the nanodisc comprising an olfactory receptor protein manufactured by the method of the present invention was measured using DLS and FE-SEM, and as a result, it was confirmed that the particle size is in the range of 10 nm to 50 nm, and the particle size of the nanodisc was assembled by itself within a certain range.

Additionally, to confirm the superiority of the present invention, cell-based and nanodisc-based functional analyses were performed with regard to the olfactory receptor which selectively senses with cadaverine, and thereby it was confirmed that the functions of the receptors are correctly maintained in the nanodisc.

The method for manufacturing a nanodisc including an olfactory receptor protein using E. coli is proceeded such that an olfactory receptor protein is produced in E. coli cells transformed with the olfactory receptor protein, and E. coli is lysed and released to the outside of the cells, and mixed with a membrane protein and reassembled in the form of a nanodisc, and therefore, a nanodisc including an olfactory receptor protein can be stably and efficiently produced.

Additionally, the nanodisc including an olfactory receptor protein implements the binding site with the detection material and thereby exhibits the effects of improving selectivity, accuracy, and reproducibility.

### BRIEF DESCRIPTION OF THE DRAWINGS

FIG. 1 shows a schematic diagram illustrating the structure of a nanodisc by the present invention.

FIG. 2 shows the results of gel staining and western blot analysis of the purified ApoA-I.

FIG. 3 shows the results of gel staining and western blot analysis of the purified TAAR13c.

FIG. 4 shows the results of western blot analysis of the TAAR13c expressed in HEK-293 cells.

FIG. 5 shows the results of cell-based analysis of the TAAR13c response to concentration-dependent treatment of CV (*p<0.05, *p<0.01, *p<4.001).

FIG. 6 shows the results of cell-based analysis for confirming the selectivity of TAAR13c against CV (HA, hydroxylamine; EA, ethanolamine; PT, putrescine; CV, cadaverine; DD, diaminodecane; TMA, trimethylamine; TEA, trimethylamine; ThiA, thiamine; TryA, tryptamine; Glu, glutamine).

FIGS. 7A to 7F show the results confirming the optimization conditions for the formation of T13NDs.

FIG. 8 shows the measurement results of the size of optimized T13NDs using DLS.

FIG. 9 shows an image of T13NDs photographed using FE-SEM.

FIG. 10 shows the results of purification of produced T13NDs by size exclusion chromatography.

FIG. 11 shows the real-time measurement results of tryptophan fluorescence of T13ND with increasing concentration of CV.

FIG. 12 shows the results of the selective response of T13ND against CV.

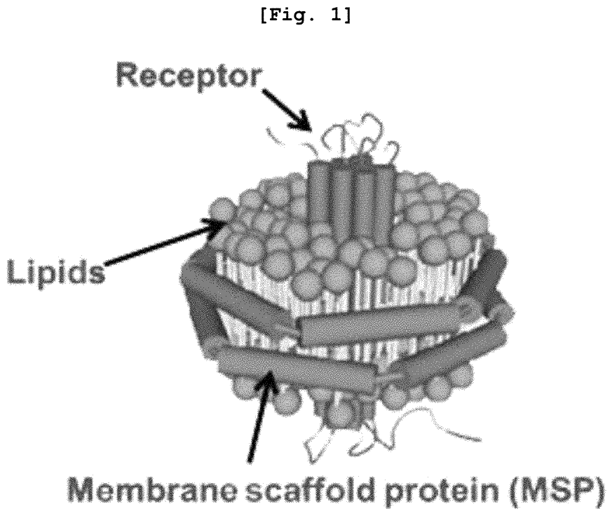

### DESCRIPTION OF EXEMPLARY EMBODIMENTS

Hereinafter, the present invention will be described in more detail with reference to Examples. Since these Examples are only for illustrating the present invention, the scope of the present invention is not construed as being limited by these Examples.

### Example 1: Cloning of ApoA-I and TAAR13c Genes

To express ApoA-I and TAAR13c proteins in E. coli, first, the ApoA-I and TAAR13c genes were cloned.

Specifically, the ApoA-I gene was designed to include 6×His and stop codon gene, and the gene was amplified by PCR using human genomic cDNA (ApoA-I forward primer (SEQ ID NO: 1): 5′ CAC CAG GAG ATA TAC ATA TGA AAG CTG CGG TGC TGA CC 3′, ApoA-I reverse primer (SEQ ID NO: 2): 5′ CTA GTG GTG GTG GTG GTG GTG CTG GGT GTT GAG CTT CTT AGT GTA 3′).

The TAAR13c gene was amplified by PCR using zebrafish DNA (TAAR13c forward primer-1 (SEQ ID NO: 3): 5′-CAC CAG GAG ATA TAC ATA TGA TGC CCT TIT GCC ACA AT 3′, TAAR13c reverse primer-1 (SEQ ID NO: 4): 5′ TGA ACT CAA TTC CAA AAA TAA TIT ACA C-3′). The amplified PCR product was inserted into the pET-DEST42 vector (Invitrogen, USA) using the gateway cloning system (Invitrogen, USA) to prepare the pET-DEST42/TAAR13c vector.

The TAAR13c gene was also inserted into to pcDNA3, a mammalian expression vector, using the amplified PCR product (TAAR13c forward primer-2 (SEQ ID NO: 5): 5′ ATG AAT TCA TGG ATT TAT CAT CAC AAG AAT 3′, TAAR13c reverse primer-2 (SEQ ID NO: 6): 5′ ATC TCG AGT CAA ACC GTA AAT AAA TTG ATA 3′).

### Example 2: Expression and Purification of ApoA-I in E. coli

BL21 (DE3) E. coli cells with the structure of pET-DEST42/ApoA-I was cultured in Luria-Bertani (LB) medium (+50 μg/mL ampicillin) and they were grown until the OD₆₀₀ value reached 0.5. Additionally, isopropyl thiogalactoside (IPTG) was added thereto at a final concentration of 1 nM to induce the overexpression of ApoA-I.

After 3 hours, the cells were centrifuged (7,000 g, 4° C., 20 min), resuspended in a lysis buffer (20 mM Tris-HCl, 0.5 M NaCl, 20 mM imidazole, pH 8.0), and disrupted by subjecting to sonication (5 s on/off, 5 min).

The disrupted cell lysate was centrifuged (12,000 g, 4° C., 30 min), and the ApoA-I in the supernatant was collected, and loaded into the HisTrap HP column (GE Healthcare, Sweden) through FPLC (GE Healthcare).

Then, the column was washed with a wash buffer (20 mM Tris-HCl, 50 mM imidazole, 0.5 M NaCl, pH 8.0), and ApoA-I was separated using a separation buffer (20 mM Tris-HCl, 400 mM imidazole, 0.5 M NaCl, pH 8.0), and dialyzed using the HEPES buffer I (20 mM HEPES-NaOH, 100 mM NaCl, 20 mM cholate, 1 mM EDTA, pH 8.0) using HiTrap HP desalting column (GE Healthcare, sweden). The dialyzed protein was stored at 4° C. until use.

### Example 3: Expression and Purification of TAAR13c

The BL21 (DE3) cells transformed with the pET-DEST42/TAAR13c vector were cultured at 37° C. until the OD₆₀₀ value reached 0.5 using the LB medium (+50 μg/mL ampicillin). The expression of TAAR13c was induced by adding 1 mM IPTG thereto and the cells were cultured for 4 hours.

After the culture, the cells were centrifuged (7,000 g, 4° C., 20 min), and the obtained pellet was resuspended in PBS containing 2 mM EDTA. Then, the cells were subjected to sonication (5 s on/off, 5 min) and again centrifuged (12,000 g, 4° C., 20 min).

After repeating sonication and centrifugation, the pellet of the sample was dissolved with a dissolution buffer (0.1 M Tris-HCl, 20 mM sodium dodecyl sulfate (SDS), 100 mM dithiothreitol (DTT), 1 mM EDTA, pH 8.0). The dissolved protein was dialyzed with 0.1 M sodium phosphate, a buffer solution containing 10 mM SDS, using the 10K MWCO dialysis cassette (Thermo Scientific, USA).

Then, the dialysate was filtered using 0.2 μm bottle top filter (Thermo Scientific, USA) and applied to the HisTrap HP column, which was equilibrated with 0.1 M sodium phosphate (pH 8.0) containing 10 mM SDS. The column was continuously washed using a wash buffer (0.1 M sodium phosphate, 10 mM SDS) until it reached pH 7.0 from pH 8.0. Then, TAAR13c was separated by dissolving with the same buffer (pH 6.0).

The separated protein by dissolution was dialyzed with the HEPES buffer II (20 mM HEPES-NaOH, 100 mM NaCl, 25 mM cholate, 1 mM EDTA, pH 8.0). The purified TAAR13c by dialysis was analyzed by SDS-PAGE and western blot analysis.

### Experimental Example 1: Western Blot Analysis and Analysis of Total Protein

The samples (20 μL) of ApoA-I and TAAR13c proteins obtained in Example 2 and Example 3 were analyzed by SDS-PAGE and western blotting.

The western blot analysis was performed using anti-FLAG rabbit Ab (Cell Signaling Technology, USA), anti-His-probe mouse Ab (Santa Cruz Biotechnology, USA), and anti-V5 epitope mouse Ab (Santa Cruz Biotechnology, USA) as primary antibodies. HRP-conjugated anti-rabbit Ab (Millipore, USA) and HRP-conjugated anti-mouse Ab (Millipore, USA) were used as secondary antibodies, and Luminata Forte western HRP substrate (Millipore, USA) was also used. The protein concentration was measured using the BCA assay kit (Pierce, Ill., USA). Specifically, the protein was electrophoresed by the SDS-PAGE method, and the protein was transferred onto a nitrocellulose blotting membrane using the tans-blot. Then, the membrane which the protein was transferred onto was subjected to membrane blocking, washed after treatment with primary antibody, and washed after treatment with secondary antibody in this order, and detected using the HRP substrate.

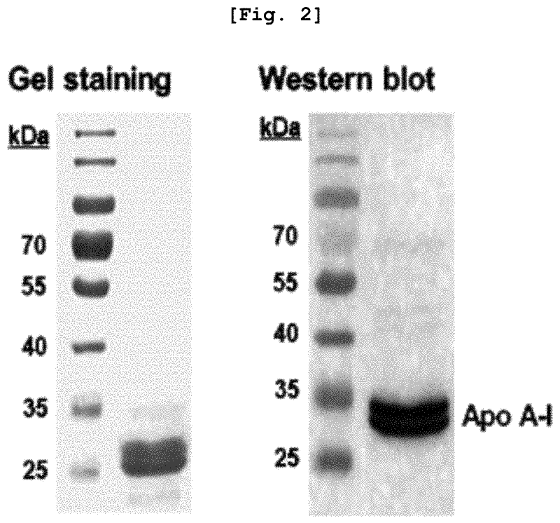

As a result, as can be seen in FIG. 2 which illustrates the gel staining results by SDS-PAGE, the ApoA-I band was distinctively confirmed. Additionally, even in the western blotting analysis using the His-Probe antibody, the ApoA-I band was distinctively confirmed. This confirms that the ApoA-I purified in Example 2 was in high purity.

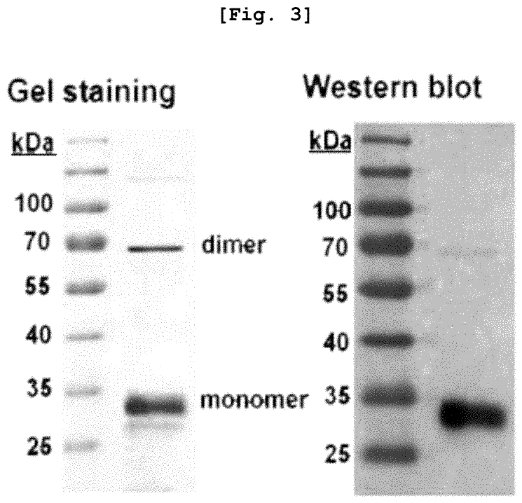

Additionally, as can be seen in FIG. 3, the TAAR13c band was distinctively confirmed in the gel staining using electrophoresis, and even in the western blotting analysis using the V5 epitope antibody, the TAAR13c band was distinctively confirmed. This confirms that the TAAR13c purified in Example 3 was in high purity.

### Example 4: Confirmation of TAAR13c Expression in HEK-293 Cells

Human embryonic kidney (HEK)-293 cells were cultured in Dulbecco's Modified Eagles Medium (DMEM) (HyClone, USA) containing 1% penicillin, 1% streptomycin (Gibco, USA) and 10% Fetal Bovine Serum (FBS)(Gibco, USA), under the conditions of 37° C. and 5% C02.

Transfection was performed by the following method using Lipofectamine® 3000. Specifically, the cells were transfected with a DNA mixture containing TAAR13c, pCRE-Luc, pSV40-RL, G_{αolf}, and Receptor-transporting protein 1 short (RTP1S) using Lipofectamine® 3000.

Then, the transfected cells were collected using phosphate-buffed saline and the cells were disrupted by sonication (2 s on/off, 2 min) (Sonics Vibracell, USA).

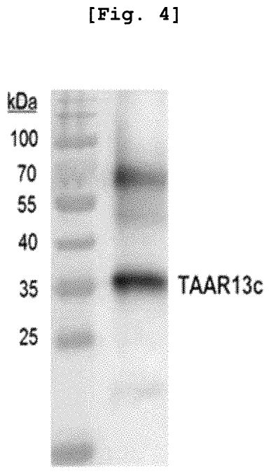

As such, the TAAR13c expressed in HEK-293 cells was detected by western blotting analysis and the results are shown in FIG. 4.

### Experimental Example 2: Confirmation of Characteristics of TAAR13c Against Cadaverine

The characteristics against cadaverine were confirmed using the TAAR13c produced in Example 4 by the Dual-Glo Luciferase assay system.

Specifically, the transfected cells were cultured in DMEM medium (50 μL) for 30 minutes, and an odorant (25 μL) designed to the desired concentration was added thereto, and cultured for 4 hours. Then, the Dual-Glo Luciferase reagent (20 μL) was added thereto, cultured at room temperature for 10 minutes, and the firefly luciferase luminescence was measured using the luminescence plate reader. Then, the Dual-Glo Stop-n-Glo reagent (20 μL) was added to the measured sample, and the mixture was cultured at room temperature for 10 minutes, and the Renilla luciferase luminescence was measured. The measured data was analyzed using the following formula. The solution without amine was used as the negative control and 10 μM forskolin (FSK) was used as the positive control:

[CRE/Renilla(N)−CRE/Renilla(0)]/[CRE/Renilla(FSK)−CRE/Renilla(0)].

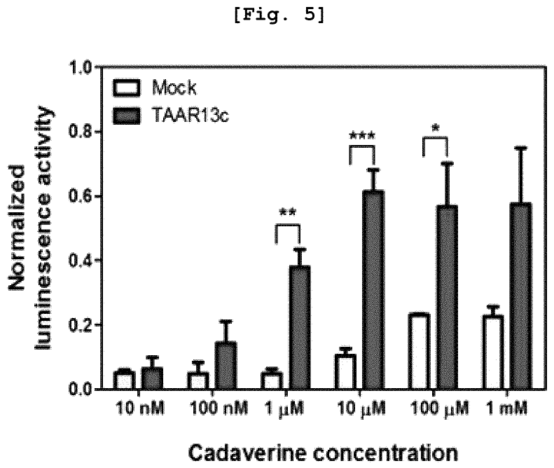

As a result, as shown in FIG. 5, the TAAR13c-expressing cells showed a significant response to CV, whereas the cells transfected with a random vector (MocK) (i.e., Comparative Example) did not show any significant response. These results indicate that TAAR13c is successfully expressed in HEK-293 cells.

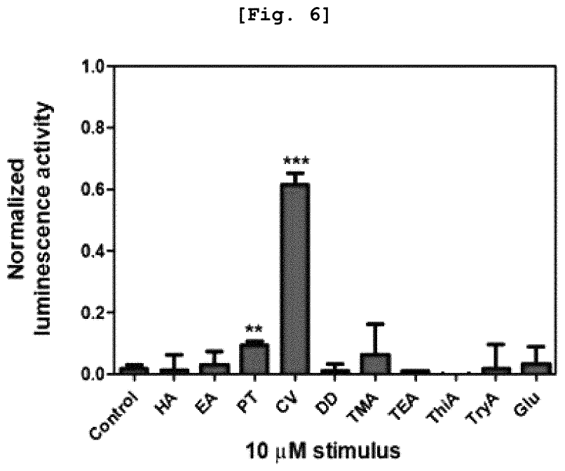

Additionally, as shown in FIG. 6, TAAR13c showed a response only against CV among the various kinds of amine molecules having different amine residues and structures, and this result shows that TAAR13c selectively responds to CV among the various kinds of amines.

### Example 5: Assembly of TAAR13c-Embedded Nanodiscs (T13NDs)

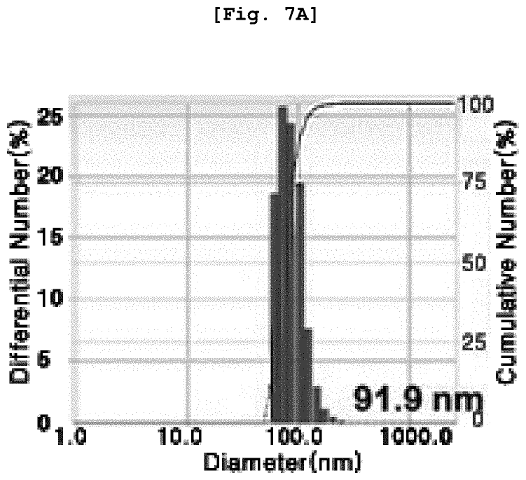

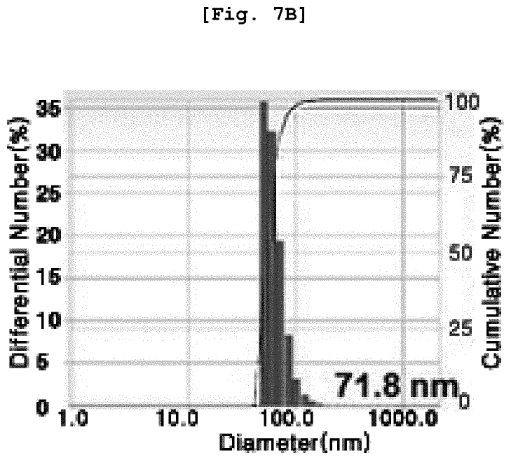

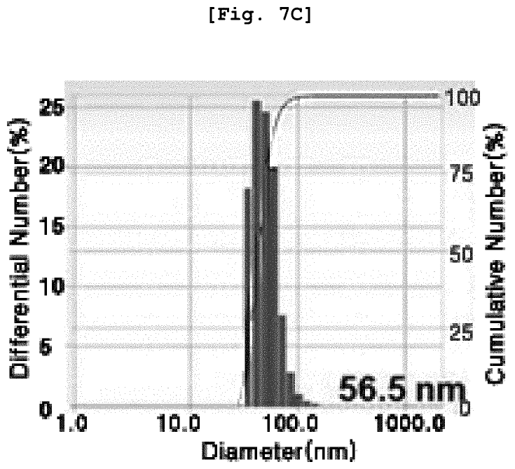

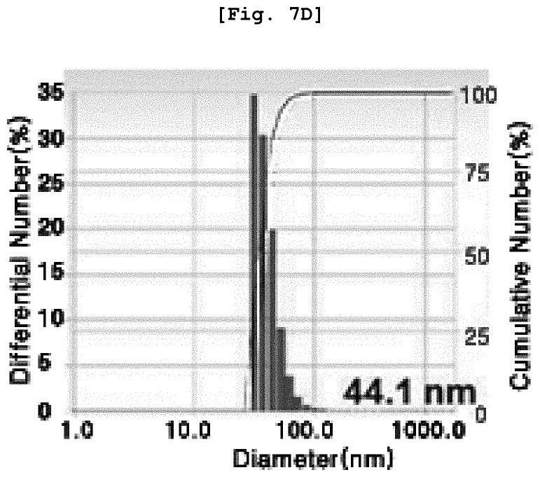

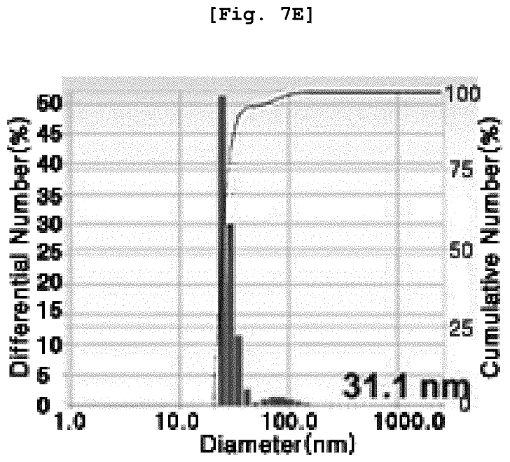

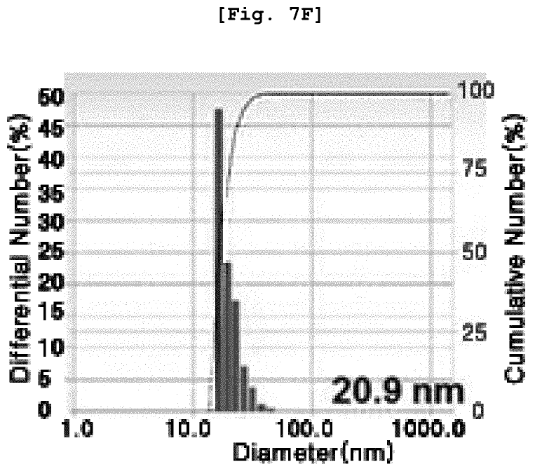

In order to determine the optimum conditions for the assembly of T13NDs before assembling T13NDs, the size of T13NDs was determined according to the lipid sonication treatment time (10 min to 60 min) and protein concentration (0.5 μM to 2 μM), and the results are shown in FIGS. 7A to 7F.

As a result, as shown in FIGS. 7A to 7F, the optimum conditions for minimizing the size of T13NDs were confirmed to be 30 minutes for the lipid sonication treatment time, 1 μM for the protein concentration, and the T13NDs were assembled using the optimum conditions.

Specifically, in order to provide an similar environment with the negative charge membrane, 1-palmitoyl-2-oleoyl-sn-glycero-3-phosphocholine (POPC) and 1-palmitoyl-2-oleoyl-sn-glycero-3-phosphoglycerol (POPG) were mixed in a 1:1 molecular rate.

Lipids were dried using nitrogen gas from a chloroform solution, and then left under vacuum for 1 hour to remove residual chloroform.

Then, the dried lipids were dissolved in HEPES buffer II, and the purified TAAR13c protein prepared in Example 3 was added thereto, and settled on ice for 10 minutes.

After settling, ApoA-I was added to the mixture, and mixed and settled while stirring at 4 C for 2 hours. The final concentration in the mixture was 1 μM for TAAR13c, 100 μM for ApoA-I, 8 mM for lipids, and 25 mM for the surfactant.

Then, in order to remove the surfactant, bio beads (Bio-Rad, USA) were added to the mixture and stirred overnight.

Finally, in order to remove unbound proteins from the mixture, size exclusion chromatography (SEC) (Superdex 200 Increase 10/300 GL, GE Healthcare, USA) was performed. The column was equilibrated with HEPES buffer III (20 mM HEPES-NaOH, 100 mM NaCl, 1 mM EDTA, pH 8.0), and the sample (500 L) was loaded into injecting loops at a speed of 0.5 mL/min using FPLC. After collecting the peak sections, the purified T13ND was stored at 4° C. before the characterization step.

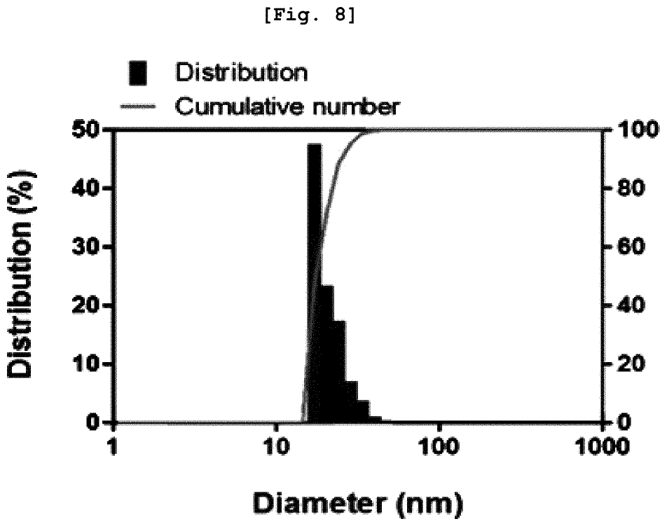

The T13NDs assembly was confirmed using the size exclusion chromatography (SEC) analysis using the prepared T13NDs solution, and as a result, as can be seen in FIG. 8, it was confirmed that nanodisc T13NDs were successfully manufactured.

### Experimental Example 3: Analysis of T13NDs

The size of the nanodisc (T13NDs) prepared in Example 5 was confirmed using dynamic light scattering spectrophotometer (DLS) (DLS-7000, Japan) and SUPRA 55VP field-emission scanning electron microscope (FE-SEM) (Carl Zeiss, Germany).

The intrinsic fluorescence of T13NDs was measured real time using the LS 55 luminescence spectrometer (Perkin Elmer, USA) (excitation 290 nm; emission 340 nm). The real-time measurement of intrinsic fluorescence of TAAR13c was measured at various amine concentrations of 1 mM to 10 mM.

As can be confirmed in FIG. 8, the size of T13NDs measured using DLS was found to be 20 nm.

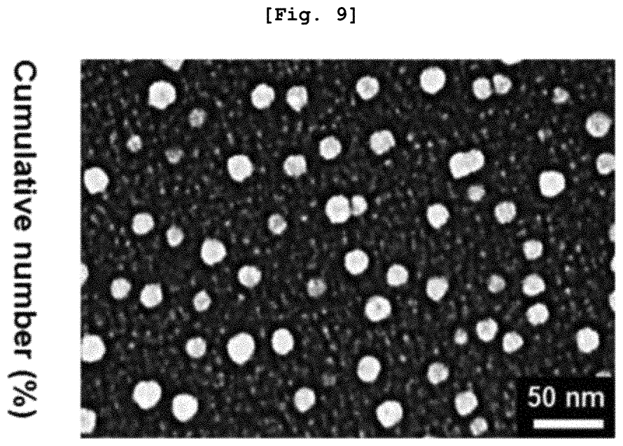

FIG. 9 shows the measured diameter in the image measured using FE-SEM, and it was confirmed to be similar to the size of the diameter measured in FIG. 5.

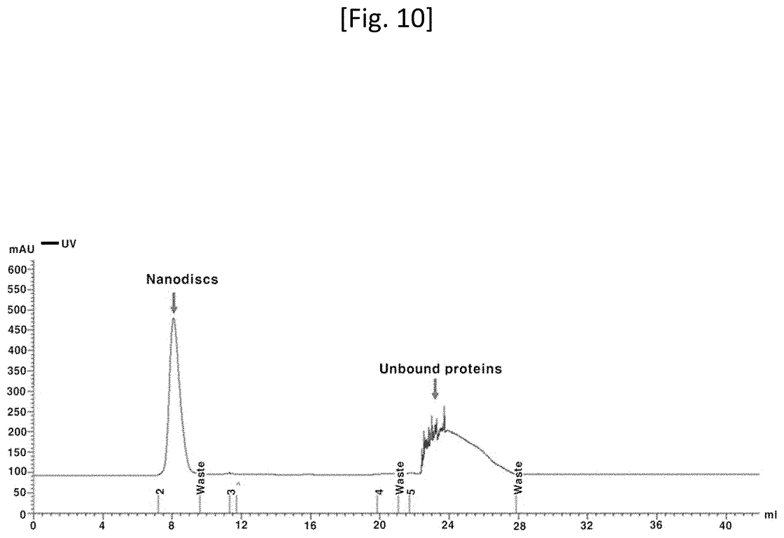

FIG. 10 shows a successful purification process of an produced olfactory receptor nanodisc using size exclusion chromatography (SEC).

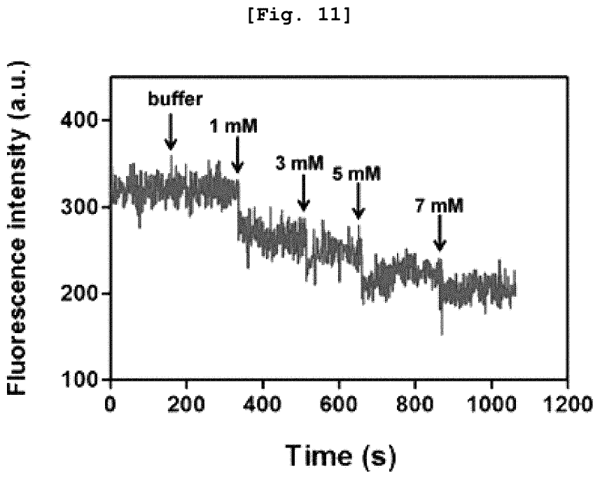

Additionally, FIG. 11 shows the measurement results of real-time tryptophan fluorescence of T13ND with increasing concentration of CV, in which tryptophan fluorescence was not measured in the control group (buffer), whereas tryptophan fluorescence was measured when treated with various CV concentrations, and these results indicate that T13ND has an affinity for CV.

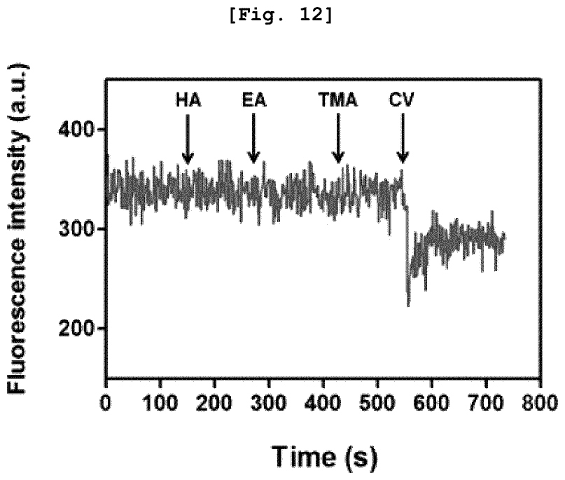

Additionally, FIG. 12 shows the results of selective response of T13ND against CV measured using the real-time tryptophan fluorescence, and the intrinsic tryptophan fluorescence of T13NDs were shown only by CV stimulus compared to various kinds of amine (HA: hydroxylamine, EA: ethanolamine, TMA: trimethylamine, CA: cadaverine) having the above amine residues and structures. These results show that TAAR13c is effectively reconstituted into nanodiscs.

In the above exemplary systems, although the methods have been described on the basis of the flowcharts using a series of the steps or blocks, the present invention is not limited to the sequence of the steps, and some of the steps may be performed at different sequences from the remaining steps or may be performed simultaneously with the remaining steps. Furthermore, those skilled in the art will understand that the steps shown in the flowcharts are not exclusive and may include other steps or one or more steps of the flowcharts may be deleted without affecting the scope of the present invention.

What is claimed is:

1. A manufacturing method of a nanodisc comprising an olfactory receptor protein, consists:
i) producing and purifying an olfactory receptor protein in E. coli;
ii) producing and purifying a membrane scaffold protein in E. coli;
iii) mixing the olfactory receptor protein, which was produced and purified in E. coli, with lipids, followed by settling;
iv) mixing the settled mixture with the membrane scaffold protein, which was produced and purified in E. coli, and stirring, thereby assembling a nanodisc.

2. The method of claim 1, wherein the producing of an olfactory receptor protein in E. coli in step i) comprises:
i-1) culturing the E. coli transformed with an olfactory receptor protein;
i-2) overexpressing an olfactory receptor protein;
i-3) lysing the E. coli and releasing the olfactory receptor protein to the outside of the cell; and
i-4) solubilizing the olfactory receptor protein, followed by separation and purification.

3. The method of claim 1, wherein the method further comprises:
v) removing surfactants and unbound proteins from the mixture of iv).

4. The method of claim 1, wherein the olfactory receptor protein produced in the E. coli is Trace amine-associated receptor 13c (TAAR13c).

5. The method of claim 1, wherein the membrane scaffold protein is apolipoprotein A-I (ApoA-I) protein, which is added so as to surround a lipid-receptor complex.

6. The method of claim 1, wherein, in step iii) where the olfactory receptor protein, which was produced in E. coli, is mixed with lipids, followed by settling, the stirring is performed at 0° C. to 10° C. for 10 minutes to 1 hour.

7. The method of claim 1, wherein, in step iii) where the olfactory receptor protein, which was produced in E. coli, is mixed with lipids, followed by settling, 1-palmitoyl-2-oleoyl-sn-glycero-3-phosphocholine (POPC) and 1-palmitoyl-2-oleoyl-sn-glycero-3-phosphoglycerol (POPG) are mixed in a 1:1 molecular rate.

8. The method of claim 1, wherein, in step iv) where the settled mixture is mixed with the membrane scaffold protein, which was produced and purified in E. coli, and stirred, thereby assembling a nanodisc, the stirring is performed for 1 hour to 2 hours.

9. A nanodisc comprising an olfactory receptor protein, which is manufactured by any one of claims 1 to 8 and has an average diameter of 15 nm to 25 nm.
